(19) United States
(12) Patent Application Publication
(10) Pub. No.: US 2019/0231730 A1
(43) Pub. Date: Aug. 1, 2019

(54) TREATMENT OF A DISEASE MEDIATED BY ARACHIDONIC ACID OR AN EICOSANOID

(71) Applicants: Robert GADAMIAN, Burbank, CA (US); Donald PLACE, Montrose, CA (US)

(72) Inventors: Robert GADAMIAN, Burbank, CA (US); Donald PLACE, Montrose, CA (US)

(21) Appl. No.: 16/319,281

(22) PCT Filed: Jul. 18, 2017
(86) PCT No.: PCT/US2017/042615

§ 371 Date: Jan. 18, 2019

Related U.S. Application Data
(60) Provisional application No. 62/363,786, filed on Jul. 18, 2016.

(51) Int. Cl. A61K 31/202; A61K 31/357; A61P 35/00; A61P 9/10; A61P 17/06; A61P 11/06; A61P 25/28; A61P 37/08; A61P 37/02; A61P 1/00

(52) U.S. Cl. CPC A61K 31/202; A61K 31/357; A61P 35/00; A61P 9/10; A61P 1/00; A61P 11/06; A61P 25/28; A61P 37/08; A61P 37/02; A61P 17/06

(57) ABSTRACT

Disclosed is a method of treating a disease mediated by arachidonic acid or an eicosanoid. The method includes administering to a subject in need thereof an effective amount of an analogue of arachidonic acid or an analogue of eicosanoid. The analogue is a dehydro-analogue of an arachidonic acid, HPETE, EET, a prostaglandin or HETE. Diseases that may be treated include cancer, ischemic heart disease, psoriasis, cystic fibrosis, Alzheimer's disease, allergy, COPD, rheumatoid arthritis (RA), ulcerative colitis, or Crohn's disease.

### CROSS-REFERENCE TO RELATED APPLICATIONS

The present invention claims priority to U.S. Provisional Application Ser. No. 62/363,786, filed Jul. 18, 2016, the entire disclosure of which, including any drawings, is incorporated by reference herein.

### FIELD OF THE INVENTION

The present invention is related to the treatment of diseases mediated by arachidonic acid or by an eicosanoid and, in particular, to treating such diseases by administering an analogue of arachidonic acid or an analogue of an eicosanoid.

### INTRODUCTION

Arachidonic acid is one of the major polyenoic fatty acids in mammals. It is the precursor of an important group of biologically active compounds, the eicosanoids that are formed by the arachidonic acid cascade. The eicosanoids thus play a major role in many different organs of the body and in the pathology of many diseases of those organs.

### SUMMARY

The present invention focuses on treatment of a disease mediated by arachidonic acid or an eicosanoid using an analogue of arachidonic acid or an analogue of an eicosanoid.

Accordingly, the present invention is directed to the treatment of diseases involving eicosanoids and arachidonic acid. The treatment may involve administering to a subject in need thereof an effective amount of a dehydro-analogue of arachidonic acid or an analogue of eicosanoid. The analogue may be an arachidonic acid analogue, a hydroperoxyeicosatetraenoic acid (HPETE) analogue, an epoxyeicosatetrienoic acid (EET) analogues, a prostaglandin analogue or hydroxyeicosatetraenoic acid (HETE) analogue.

In various embodiments, the dehydro-analogues may have one or more triple bonds replacing one or more of the double bonds in arachidonic acid, 15-HPETE, 12-HPETE, 5-HPETE, 5,6-EET, 8,9-EET, 14,15-EET, 11,12-EET, prostaglandin PGG2, prostaglandin PGH2, 5-HETE, 8-HETE, 9-HETE, 11-HETE, 12-HETE, 15-HETE, 19-HETE or 20-HETE.

In various embodiments, the disease treated may be cancer, ischemic heart disease, psoriasis, cystic fibrosis, Alzheimer's disease, allergy, chronic obstructive pulmonary disease (COPD), rheumatoid arthritis (RA), ulcerative colitis, or Crohn's disease.

### BRIEF DESCRIPTION OF THE DRAWINGS

FIG. 1 illustrates the arachidonic acid cascade.

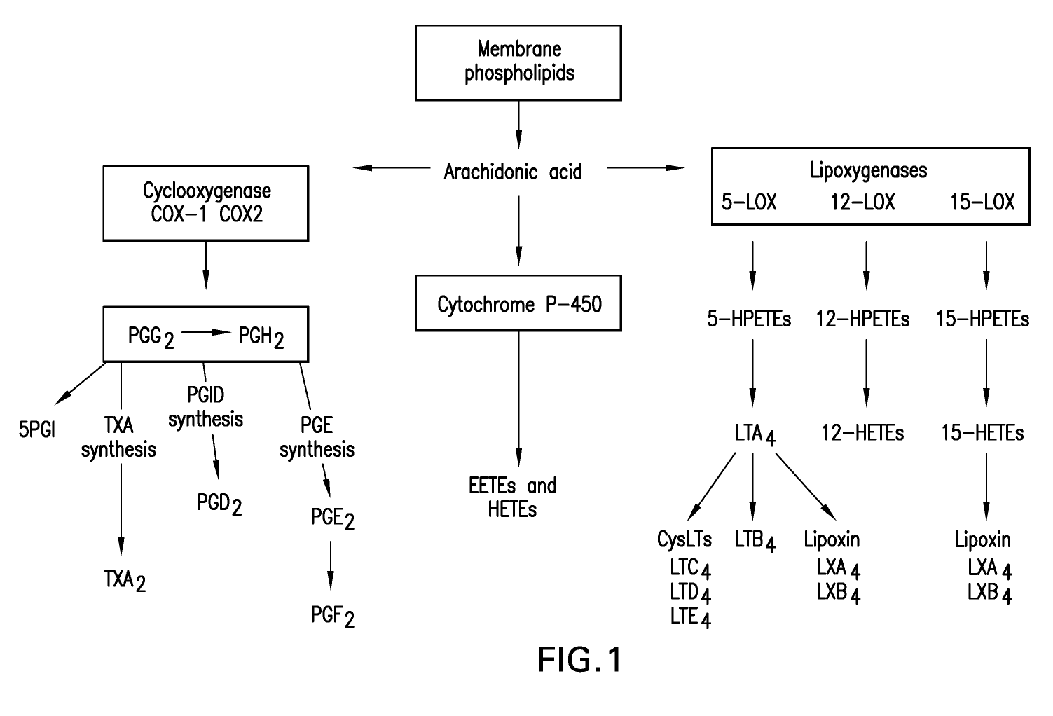

### DETAILED DESCRIPTION

The present invention is directed to the treatment of diseases mediated by arachidonic acid or an eicosanoid by administering a dehydro-analogue of arachidonic acid or a dehydro-analogue of an eicosanoid.

### Definitions

As used herein, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

As used herein, the term “about” is intended to refer to a range of values above and below a stated value such as for example, values encompassing 10% below up to 10% above a stated value.

The term “and/or” is intended to mean either or both of two recited elements.

The term “inhibitor” refers to a substance that can reduce or prevent the activity of an enzyme or enzyme system. For example, an inhibitor of a delta-6-desaturase or a delta-6-desaturase reduces, diminishes or prevents the activity of the enzyme.

The terms “substance”, “agent” or “compound” may be used interchangeably herein in connection with treating a disease or condition. The substances, agents or compounds of the present invention may be an active pharmaceutical ingredient (API) in a pharmaceutically acceptable formulation.

Reference herein to an API is intended to include pharmaceutically acceptable solvates, salts, hydrates or hydrated salts, their optical isomers, racemates, diastereomers, enantiomers or the polymorphic crystalline structures of the compounds.

The term “pharmaceutical composition” or ‘pharmaceutical preparation” refers to a composition that combines one or more API's with a pharmaceutically acceptable carrier such that the composition is suitable for therapeutic use in vitro, in vivo or ex vivo.

As used herein, the term “pharmaceutically acceptable carrier” encompasses any suitable pharmaceutical carriers, such as a phosphate buffered saline solution, water, and emulsions, such as an oil/water or water/oil emulsion, various types of wetting agents and the like. The compositions also can include stabilizers and preservatives. Examples of carriers, stabilizers and adjuvants, can be found in Remington: The Science and Practice of Pharmacy, Lippincott Williams & Wilkins, Twenty-First edition (May 19, 2005).

The terms “treatment” or “treating” as used herein, may include ameliorating, suppressing, eradicating, reducing the severity of, decreasing the frequency of incidence of, preventing, reducing the risk of, and/or delaying the onset of a disease or condition. In various embodiments, the treatment may be targeted to the underlying disease and not to disease symptoms or to ancillary pathologic processes that are not directly related to the underlying disease. In various other embodiments, the treatment may target the underlying disease, disease symptoms and ancillary pathological processes or any combination thereof.

The analogues of the present invention may be used in treating various diseases mediated by arachidonic acid or by an eicosanoid.

While not intending to be bound by a mechanism of action, it is believed that the analogues of the present invention may act by binding to active sites on enzymes to block the binding to a substrate for that enzyme thereby blocking the enzyme action and/or by binding to a receptor site to block the binding of a ligand that would otherwise bind to the receptor and elicit a biological response.

Various of the analogues as described below, may have enantiomeric forms and such analogues may be S enantiomers in various embodiments, R enantiomers in various embodiments or mixtures thereof in various embodiments.

Analagoues

Arachidonic Acid Analogues

The structure of arachidonic acid is as show below.

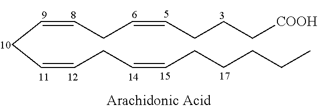 [SMILES: CCCCC/C=C\C/C=C\C/C=C\C/C=C\CCCC(=O)O]

The term “arachidonic acid analogue” as used herein, is intended to refer to a modification of the arachidonic acid structure above in which from one to three of the double bonds is a triple bond, said triple bond(s) being located at position C5-C6, C8-C9, C11-C12 or C14-C15.

Hydroperoxyeicosatetraenoic Acid (HPETE) Analogues

The structure of 15-hydroperoxyeicosatetraenoic acid (15-HPETE) is as shown below.

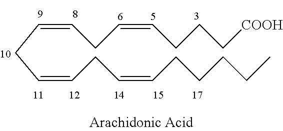

The term “15-HPETE analogue” as used herein, is intended to refer to a modification of the 15-HPETE structure above in which from one to four of the double bonds is a triple bond, said triple bond(s) being located at position C5-C6, C8-C9, C11-C12 or C13-C14.

The structure of 12-hydroperoxyeicosatetraenoic acid (12-HPETE) is as shown below.

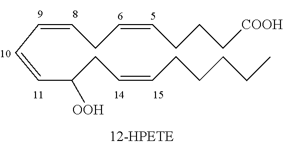

The term “12-HPETE analogue” as used herein, is intended to refer to a modification of 12-HPETE structure above in which from one to four of the double bonds is a triple bond, said triple bond(s) being located at position C5-C6, C8-C9, C10-C11 or C14-C15.

The structure of 5-hydroperoxyeicosatetraenoic acid (5-HPETE) is as shown below.

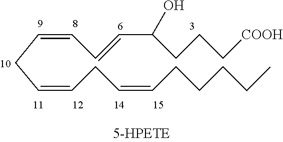

The term “5-HPETE analogue” as used herein, is intended to refer to a modification of 5-HPETE structure above in which from one to four of the double bonds is a triple bond, said triple bond(s) being located at position C6-C7, C8-C9, C11-C12 or C14-C15.

Epoxyeicosatetrienoic Acid (EET) Analogues

The structure of 5,6-epoxyeicosatetrienoic acid (5,6-EET) is as shown below.

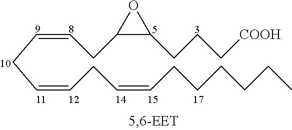 [SMILES: CCCCCC/C=C\C/C=C\C/C=C\CC1OC1CCCC(=O)O]

The term “5,6-EET analogue” as used herein, is intended to refer to a modification of 5,6-EET structure above in which from one to three of the double bonds is a triple bond, said triple bond(s) being located at position C8-C9, C11-C12 or C14-C15.

The structure of 8,9-epoxyeicosatetrienoic acid (8,9-EET) is as shown below.

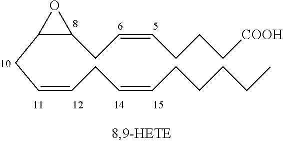 [SMILES: CCCCC/C=C\C/C=C\CC1OC1C/C=C\CCCC(=O)O]

The term “8,9-EET analogue” as used herein, is intended to refer to a modification of 8,9-EET structure above in which from one to three of the double bonds is a triple bond, said triple bond(s) being located at position C5-C6, C11-C12 or C14-C15.

The structure of 14,15-epoxyeicosatetrienoic acid (14,15-EET) is as shown below.

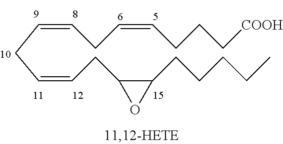 [SMILES: CCCCCC1OC1C/C=C\C/C=C\C/C=C\CCCC(=O)O]

The term “14,15-EET analogue” as used herein, is intended to refer to a modification of 14,15-EET structure above in which from one to three of the double bonds is a triple bond, said triple bond(s) being located at position C5-C6, C8-C9 or C11-C12.

The structure of 11,12-epoxyeicosatetrienoic acid (11,12-EET) is as shown below.

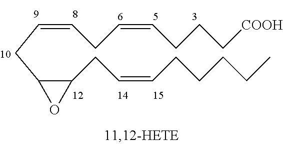 [SMILES: CCCCC/C=C\CC1OC1C/C=C\C/C=C\CCCC(=O)O]

The term “11,12-EET analogue” as used herein, is intended to refer to a modification of 11,12-EET structure above in which from one to three of the double bonds is a triple bond, said triple bond(s) being located at position C5-C6, C8-C9 or C14-C15.

Prostaglandin Analogues

The structure of Prostaglandin G2 (PGG2) is as shown below.

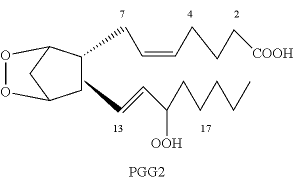

The term “PGG2 analogue” as used herein, is intended to refer to a modification of PGG2 structure above in which from one to two of the double bonds is a triple bond, said triple bond(s) being located at position C5-C6 or C13-C14.

The structure of Prostaglandin H2 (PGH2) is as shown below.

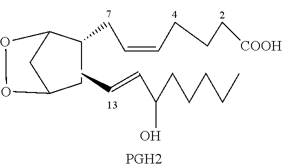

The term “PGH2 analogue” as used herein, is intended to refer to a modification of PGH2 structure above in which from one to two of the double bonds is a triple bond, said triple bond(s) being located at position C5-C6 or C13-C14.

Hydroxyeicosatetraenoic Acid (HETE) Analogues

The structure of 5-hydroxyeicosatetraenoic acid (5-HETE) is as shown below.

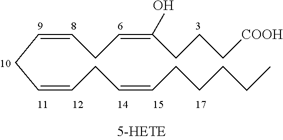

The term “5-HETE analogue” as used herein, is intended to refer to a modification of the 5-HETE structure above in which from one to four of the double bonds is a triple bond, said triple bond(s) being located at position C6-C7, C8-C9, C11-C12 or C14-C15

The structure of 8-hydroxyeicosatetraenoic acid (8-HETE) is as shown below.

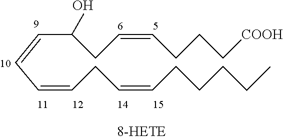 [SMILES: CCCCC/C=C\C/C=C\C=C/C(O)C/C=C\CCCC(=O)O]

The term “8-HETE analogue” as used herein, is intended to refer to a modification of the 8-HETE structure above in which from one to four of the double bonds is a triple bond, said triple bond(s) being located at position C5-C6, C9-C10, C11-C12 or C14-C15.

The structure of 9-hydroxyeicosatetraenoic acid (9-HETE) is as shown below.

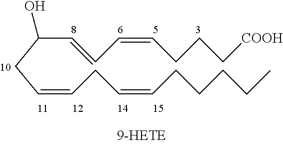 [SMILES: CCCCC/C=C\C/C=C\CC(O)/C=C/C=C\CCCC(=O)O]

The term “9-HETE analogue” as used herein, is intended to refer to a modification of the 9-HETE structure above in which from one to four of the double bonds is a triple bond, said triple bond(s) being located at position C5-C6, C7-C8, C11-C12 or C14-C15.

The structure of 11-hydroxyeicosatetraenoic acid (11-HETE) is as shown below.

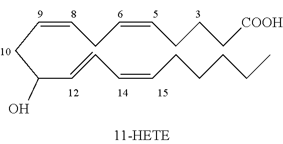 [SMILES: CCCCC/C=C\C=C\C(O)C/C=C\C/C=C\CCCC(=O)O]

The term “11-HETE analogue” as used herein, is intended to refer to a modification of the 11-HETE structure above in which from one to four of the double bonds is a triple bond, said triple bond(s) being located at position C5-C6, C8-C9, C12-C13 or C14-C15.

The structure of 12-hydroxyeicosatetraenoic acid (12-HETE) is as shown below.

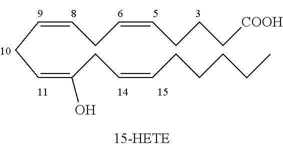 [SMILES: CCCCC/C=C\C/C(O)=C\C/C=C\C/C=C\CCCC(=O)O]

The term “12-HETE analogue” as used herein, is intended to refer to a modification of the 12-HETE structure above in which from one to four of the double bonds is a triple bond, said triple bond(s) being located at position C5-C6, C8-C9, C10-C11 or C14-C15.

The structure of 15-hydroxyeicosatetraenoic acid (15-HETE) is as shown below.

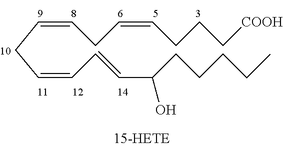 [SMILES: CCCCCC(O)/C=C/C=C\C/C=C\C/C=C\CCCC(=O)O]

The term “15-HETE analogue” as used herein, is intended to refer to a modification of the 15-HETE structure above in which from one to four of the double bonds is a triple bond, said triple bond(s) being located at position C5-C6, C8-C9, C11-C12 or C13-C14.

The structure of 19-hydroxyeicosatetraenoic acid (19-HETE) is as shown below.

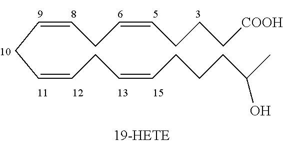 [SMILES: CC(O)CCC/C=C\C/C=C\C/C=C\C/C=C\CCCC(=O)O]

The term “19-HETE analogue” as used herein, is intended to refer to a modification of the 19-HETE structure above in which from one to four of the double bonds is a triple bond, said triple bond(s) being located at position C5-C6, C8-C9, C11-C12 or C14-C15.

The structure of 20-hydroxyeicosatetraenoic acid (20-HETE) is as shown below.

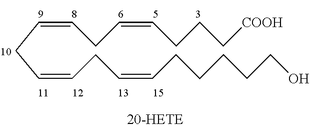 [SMILES: O=C(O)CCC/C=C\C/C=C\C/C=C\C/C=C\CCCCCO]

The term “20-HETE analogue” as used herein, is intended to refer to a modification of the 20-HETE structure above in which from one to four of the double bonds is a triple bond, said triple bond(s) being located at position C5-C6, C8-C9, C11-C12 or C14-C15.

Diseases

Cancer

In the arachidonic acid cascade, cyclooxygenase (COX) and lipoxygenase (LOX) enzymes pathways produce prostaglandins and leukotrienes, respectively, and these have been implicated in cancer (see for example Wang, D. and Dubois, R. N., Eicosanoids and cancer. Nature Reviews. Cancer, 2010, 10, 181-193.). In addition, the cytochrome P450 (P450) pathway produces eicosanoids that have also been implicated in cancer (see for example Panigrahy, D. et al., Cytochrome P450-derived eicosanoids: the neglected pathway in cancer, Cancer Metastasis Rev. (2010) 29:723-735). Thus, the inhibition of the arachidonic acid cascade by the analogues of the present invention decreases the production of COX, LOX and P450 pathway eicosanoids thus providing a method of treatment of cancer.

Eicosanoids in Cancer

Among prostanoids, proinflammatory PGE2 has a predominant role in promoting tumour growth (Nat Rev Cancer. 2010 March; 10(3): 181-193). PGE2 is the most abundant prostaglandin that is found in various human malignancies, including colon, lung, breast, and head and neck cancer, and is often associated with a poor prognosis. Multiple lines of evidence from mouse models of colorectal cancer (CRC) demonstrate that COX2-derived PGE2 promotes tumour growth. PGE2 treatment blocks NSAID-induced regression of small intestinal adenomas in ApcMin/+ mice and increased endogenous PGE2 levels through the loss of 15-PGDH inhibit the anti-tumour effects of celecoxib in the azoxymethane (AOM) mouse model. Direct evidence that PGE2 promotes tumour growth comes from recent studies showing that PGE2 treatment dramatically increased both small and large intestinal adenoma burden in ApcMin/+ mice and significantly enhanced AOM-induced colon tumour incidence and multiplicity. Furthermore, increased endogenous PGE2 through the genetic deletion of 15-Pgdh promotes colon tumour growth in ApcMin/+ and AOM mouse models. By contrast, inhibition of endogenous PGE2 through the genetic deletion of prostaglandin E synthase (Ptges) suppresses intestinal tumorigenesis in ApcMin/+ and AOM models. The central role of PGE2 in colorectal tumorigenesis has been further confirmed by evaluating mice with a Wang and DuBois homozygous deletion of individual PGE2 receptors.

LTB4 levels are increased in human colon and prostate cancer, and the expression of LTB4 receptors is increased in human pancreatic cancer. LTB4 expression is also increased in HRAS-v12-transformed cells and the receptor BLT2 is required for Ras-induced transformation in vivo. Furthermore, inhibition of LTB4 synthesis by treatment with an LTA4 hydrolase inhibitor, bestatin, reduced the burden of oesophageal adenocarcinoma in a rat model.

The CysLT1 receptor is highly expressed in human colon and prostate cancers and negatively correlates with patient survival. Increased CysLT1 expression in CRC correlates with the ability of LTD4 to induce proliferation and inhibit apoptosis. By contrast, reduced expression of the CysLT2 receptor is associated with a poor prognosis in patients with CRC, and CysLT2 signaling is involved in inducing apoptosis and terminal differentiation.

Thus, the inhibition of COX and LOX enzymes and pathways in the arachidonic acid cascade by arachidonic acid analogues or eicosanoids analogues in the present invention provides a method of treatment of cancer.

Ischemic Heart Disease

A number of studies have found a positive correlation between the amount of arachidonic acid in adipose tissue and the risk of ischemic heart disease (Lang, P D, et al., Fatty acid composition of adipose tissue, blood, lipids, and glucose tolerance in patients with different degrees of angiographically documented coronary arteriosclerosis, Res. Exp. Med., 1982, 180: 161-168). The arachidonic acid level has been correlated with body mass index in adipose tissue a well-known risk factor for myocardial infarction (Baylin, A and Campos, H., Arachidonic acid in adipose tissue is associated with nonfatal myocardial infarction in the central valley of Costa Rica. J. Nutr., 2004 134: 3095-3099; see also Hjelte, L E and Nilsson, A, Arachidonic Acid and Ischemic Heart Disease, American Society for Nutritional Sciences. J. Nutr., 2005, 135: 2271-2273, 2005).

Thus, the inhibition of receptors in the arachidonic acid cascade by arachidonic acid analogues or eicosanoids analogues in the present invention provides a method of treatment of ischemic heart disease.

Psoriasis

Eicosanoids, including prostaglandins, thromboxane, leukotrienes and hydroxyeicosatetraenoic acids, have been implicated in pathological conditions of human skin. In particular, a number of phenomena observed in psoriasis may be mediated by the action of eicosanoids (Ikai K, Psoriasis and the arachidonic acid cascade, J Dermatol Sci. 1999 November; 21(3):135-46).

Thus, the inhibition of receptors in the arachidonic acid cascade by arachidonic acid or eicosanoids analogues in the present invention provides a method of treatment of Psoriasis.

Cystic Fibrosis

The defective regulation of arachidonic acid in cystic fibrosis is believed to be the basic defect causing the disease (Carlstedt-Duke, J. et al. Pathological regulation of arachidonic acid release in cystic fibrosis: The putative basic defect, Proc. Natl. Acad. Sci. USA 1986, 83: 9202-9206). It has been shown that there is a decrease in linoleic acid and an increase in arachidonic acid in cystic fibrosis which is consistent with an activation of desaturases and elongases, enzymes that convert linoleic acid to downstream (n-6) fatty acid metabolites (Ollero M et al., Evidence of increased flux to n-6 docosapentaenoic acid in phospholipids of pancreas from cftr 2/2 knockout mice. Metabolism. 2006; 55:1192-200).

Recent studies have uncovered an underlying biochemical mechanism for some of these changes, namely increased expression and activity of fatty acid desaturases. Among other effects, this drives metabolism of linoeate to arachidonate. Increased desaturase expression appears to be linked to cystic fibrosis mutations via stimulation of the AMP-activated protein kinase in the absence of functional CFTR protein. There is evidence that these abnormalities may contribute to disease pathophysiology by increasing production of eicosanoids, such as prostaglandins and leukotrienes, of which arachidonate is a key substrate (Int J Mol Sci. 2014 September; 15(9): 16083-16099).

Patients and models of cystic fibrosis (CF) exhibit consistent abnormalities of polyunsaturated fatty acid composition, including decreased linoleate (LA) and docosahexaenoate (DHA) and variably increased arachidonate (AA), related in part to increased expression and activity of fatty acid desaturases (J Lipid Res. 2012 February; 53(2): 257-265).

Thus, the inhibition of receptors in the arachidonic acid cascade by arachidonic acid analogues or eicosanoids analogues in the present invention provides a method of treatment of cystic fibrosis.

Alzheimer's Disease

Two polyunsaturated fatty acids are predominate in the brain: arachidonic acid and DHA. The enzymes that release fatty acids are a group called phospholipases A2 (PLA2). Studies have suggest that aberrant action of the arachidonic-acid-selective PLA2 is associated with Alzheimer (Sanchez-Mejia et al., Phospholipase A2 and Arachidonic Acid in Alzheimer's Disease, Biochim Biophys Acta. 2010; 1801(8): 784-790; Rapoport S, Arachidonic Acid and the Brain, J. Nutr., 2008, 138: 2515-2520).

Thus, the inhibition of receptors in the arachidonic acid cascade by arachidonic acid analogues or eicosanoids analogues in the present invention provides a method of treatment of Alzheimer's Disease.

Allergy

An imbalance in dietary intake of essential fatty acids such as omega-3 polyunsaturated fatty acids and omega-6 polyunsaturated fatty acids may lead to a predisposition to allergic disease. This is usually caused by an increased intake of n-6 fatty acids, such as linoleic acid, with a simultaneously decreased intake of omega-3 fatty acids, such as docosahexanoic acid (DHA, C22:6), eicosapentanoic acid (EPA, C20:5) and docosapentanoic acid (DPA, C22:5) (Gottrand, F. Long-chain polyunsaturated fatty acids influence the immune system of infants. J. Nutr. 2008, 138, 1807s-1812s). Arachidonic acid has been shown to be a potent simulator of allergic cascade in a cell line model (Ahmed, N et al., Exploring the Effects of Omega-3 and Omega-6 Fatty Acids on Allergy Using a HEK-Blue Cell Line, Int. J. Mol. Sci. 2016, 17: 220;).

Thus, the inhibition of receptors in the arachidonic acid cascade by arachidonic acid analogues or eicosanoids analogues in the present invention provides a method of treatment of allergy.

Asthma

An increase in arachidonic acid has been shown to occur in cells from patients with bronchial asthma (Calabrese, C et al., Arachidonic acid metabolism in inflammatory cells of patients with bronchial asthma, Allergy 2000, 55: Suppl 61: 27±30).

Thus, the inhibition of receptors in the arachidonic acid cascade by arachidonic acid analogues or eicosanoids analogues in the present invention provides a method of treatment of asthma.

Rheumatoid Arthritis (RA)

Rheumatoid arthritis (RA) is an autoimmune articular disease associated with chronic inflammation of the joints. The most prominent mediators known to play a pivotal role in initiation and progression of articular inflammation during RA include proinflammatory cytokines, such as tumor necrosis factor-α and interleukin-1, reactive oxygen radicals, growth factors and lipid mediators generated via arachidonic acid (AA) metabolism. A diverse range of lipid mediators generated via AA biosynthetic metabolism i.e., eicosanoids mediate a wide variety of physiological and pathological functions. Eicosanoids play a central role in the progression and chronic inflammation associated with RA by mediating proinflammatory actions specially PGE2 and LTB4 (Arachidonic acid-derived eicosanoids in rheumatoid arthritis: implications and future targets. Future Rheumatology June 2006, Vol. 1, No. 3, Pages 323-330).

Thus, the inhibition of receptors in the arachidonic acid cascade by arachidonic acid analogues or eicosanoids analogues in the present invention provides a method of treatment of RA.

Chronic Obstructive Pulmonary Disease (COPD)

The potential for leukotrienes as mediators to target in the development of novel therapies for diseases such as COPD is underscored by their inflammatory behavior and the capacity of leukotriene receptor antagonists and synthesis inhibitors to reduce inflammatory responses when administered in vivo. Airway neutrophilia in COPD patients is believed to be a contributing source of inflammation and is associated with airway remodeling. The presence of neutrophils is mediated in part by leukotriene B(4) (LTB(4)), and the capacity for LTB(4) alone to replicate many aspects of neutrophilic inflammation has provided the focus of drug development toward its specific antagonism. More recently, the potential involvement of the monocyte-macrophage lineage in the etiology of COPD has received growing attention as a target for leukotriene inhibition (Chest. 2002 May; 121(5 Suppl):197S-200S).

Leukotriene B4, (LTB 4), a neutrophil and T cell chemoattractant which is produced by macrophages, neurophils and epithelial cells, is a potent inflammatory mediator. Also cysteinyl leukotrienes (LTC4, LTD4 and LTE4) are known to induce mucus secretion, inflammatory cell infiltration, increase vascular permeability and tissue edema, damage ciliary clirens, and cause severe bronchoconstriction. These are derivatives of arachidonic acid, metabolized via 5-lypoxygenase (5-LO) pathway. There are several sites along this pathway that antileukotriene agents exert their action and at the end-organ receptors (Inflamm Allergy Drug Targets. 2009 September; 8(4):297-306).

Thus, the inhibition of receptors in the arachidonic acid cascade by arachidonic acid analogues or eicosanoids analogues in the present invention provides a method of treatment of COPD.

Ulcerative Colitis, Crohn's Disease

There is a growing evidence that dietary fatty acids involved in the etiology of ulcerative colitis (UC). Arachidonic acid, an n-6 polyunsaturated fatty acid, is a precursor of the proinflammatory cytokines prostaglandin E2 and leukotriene B4. Dietary arachidonic acid has been positively associated with UC development and oleic acid which is an inhibitor of leukotriene B4, was inversely associated with associated with UC development (See Eur J Gastroenterol Hepatol. 2014 January; 26(1):11-8.).

Thus, the inhibition of receptors in the arachidonic acid cascade by arachidonic acid analogues or eicosanoids analogues in the present invention provides a method of treatment of Ulcerative Colitis and Crohn's disease.

### EXAMPLES

The following examples have not been performed, but are provided to illustrate the tests that could be performed to show activity of the analogues of the present invention.

These example illustrates in vitro testing to measure the inhibition of the enzymes in the arachidonic acid cascade.

There are various in-vitro tests for measurement of the inhibition of the enzymes in the COX, LOX and P-450 pathways. One test for each pathway is presented here.

### Example 1. In-Vitro Test for Measurement of the Enzymes in the COX Pathway

Inhibition of the enzymes in the COX pathway can be tested using the COX colorimetric inhibitor screening assay kit No. 701050 (Cayman Chemical, 1180 East Ellsworth Road, Ann Arbor, Mich. 48108 USA as described at https://www.caymanchem.com/product/701050).

### Example 2. In-Vitro Test for Measurement of the Enzymes in the LOX Pathway

Material and Methods,

Chemicals.

3-Methyl-2-benzothiazolinone (MBTH), 3-(di-methylamino)benzoic acid (DMAB), hemoglobin (bovine), hematin (porcine), linoleic acid (free acid), Tween 20, and sodium lauryl sulfate are used.

Preparation of Solutions.

Stock solutions of 10 mM MBTH and 5 mg/mL hemoglobin (100 their final concentrations) are made by dissolving the reagents in water. These solutions are stable for several weeks in a refrigerator. Hematin at 0.1 mg/mL is prepared by dissolving 2 mg in 1 mL of 0.1 N NaOH, then diluting with 19 mL of water. A solution containing 20 mM DMAB and 100 mM phosphate buffer is prepared by dissolving 330 mg DMAB in 5 mL of 1 N HCl. This is then diluted to about 80 mL with water and 1.42 g of Na2HPO is added. The pH is adjusted to 6.0 with HCl and the volume brought to 100 mL with water.

Linoleic acid substrate is prepared as described by Axelrod et al. (1), except that the ratio of Tween 20 to linoleic acid is increased from 1:1 to 2:1 (w/w). At the assay pH of 6, a 1:1 ratio of detergent to lipid produced a turbid assay solution when linoleic acid is added at 0.5 mM. Increasing the Tween 20 to a 2:1 ratio eliminated the cloudiness. A 25 mM stock solution is prepared by adding 155 μL (140 mg) of linoleic acid and 257 μL (280 mg) of Tween 20 to 5 mL of water. The mixture is emulsified by drawing back and forth in a Pasteur pipet, then clarified by adding 0.6 mL of 1 N NaOH. After dilution to the final volume of 20 mL, the solution is divided into 1 mL aliquots which are flushed with N and stored at −20° C. Linoleic hydroperoxide used as a standard is prepared enzymatically as described in Gibian and Vanderberg (2). The concentration of this standard is determined from its absorbance at 235 nm in pH 9 borate buffer assuming an extinction coefficient of 23 000 M−1cm−1.

Reaction of Linoleic Hydroperoxide.

The reaction of linoleic hydroperoxide with the colorimetric reagents is carried out in a solution containing 5 mM DMAB, 0.1 mM MBTH, 50 mM phosphate buffer (pH 6.0), and either 50 μg/mL hemoglobin or 1 μg/mL hematin in a final volume of 1.0 mL. Reactions are initiated by the addition of 10 μL of a 4.1 mM solution of linoleic hydroperoxide in ethanol, and absorbance at 590 nm is monitored for 15 min. For the standard curve, 50 μg/mL hemoglobin is used and the amount of added linoleic hydroperoxide varies.

The reaction is allowed to proceed for 30 min prior to reading absorbances at 590 nm.

Preparation of Plant Extracts. Plant material can be purchased at a local market. An enriched lipoxygenase is prepared from red-skinned potatoes by ammonium sulfate fractionation as described by Aziz et al. (3). The 45% ammonium sulfate pellet is resuspended in 100 mM phosphate buffer (pH 6) to approximately 5 mg/mL protein, divided into aliquots, and stored at −20° C. Crude homogenates are prepared by grinding either 20 g of tomato, 10 g of corn, or 5 g of peas, green beans, and potatoes in 50 mL of ice cold water for 30 s in a Waring blender, then filtering the homogenate through 4 layers of cheesecloth.

Lipoxygenase Assay Conditions. All assays should be performed at room temperature (23° C.). Initially, assay conditions involved incubating a sample in 50 mM phosphate buffer (pH 6.0) and 0.5 mM linoleic acid (plus Tween 20), then adding DMAB, MBTH, and hemoglobin to determine the amount of product formed. A simplified standard assay procedure using two working solutions should be prepared from the stock solutions described above. Solution A is prepared by mixing 10 mL of the 20 mM DMAB, 100 mM phosphate buffer solution (pH 6), 0.4 mL of the 25 mM linoleic acid stock, and 9.6 mL of water. Solution B is prepared by mixing 0.4 mL of 10 mM MBTH, 0.4 mL of 5 mg/mL hemoglobin, and 19.2 mL water. For the standard two-step assay, the sample, in a volume of 2 to 10 μL, should be incubated with 0.5 mL of solution A. After incubation for the specified amount of time (generally 5 min), 0.5 mL of solution B is added. After an additional 5 min, 0.5 mL of 1% (w/v) sodium lauryl sulfate is added to terminate the reaction. Absorbance at 598 nm was then determined.

### REFERENCES

1. Axelrod, B.; Cheesebrough, T. M.; Laakso, S. Lipoxygenase from soybeans. Methods Enzymol. 1981, 71, 441451.
2. Gibian, M. J.; Vandenberg, P. Product yield in oxygenation of linoleate by soybean lipoxygenase: the value of the molar extinction coefficient in the spectrophotometric assay. Anal. Biochem. 1987, 163, 343-349.
3. Aziz, S.; Wu, Z.; Robinson D. S. Potato lipoxygenase catalysed co-oxidation of â-carotene. Food Chem. 1999, 64, 227-230.

### Example 3. In-Vitro Test for Measurement of the Enzymes in the P-450 Pathway

The Promega P450-Glow™ kit for screening for Cytochrome P450 activity by a Luminescent assay can be used to measure the activity of enzymes in the P450 pathway (Promega Corp., 2800 Woods Hollow Road, Madison, Wis. 53711-5399 USA, as described at http://www.promega.jp/˜/media/files/resources/cell%20notes/cn013/screen%20for%20cytochrome%20p450%20activity %20using%20a %20luminescent%20assay.pdf?la=ja-jp see also https://www.promega.com/˜/media/files/resources/protocols/technical %20bulletins/101/p450%20glo%20assays%20protocol.pdf).

As various changes could be made in the above methods and compositions without departing from the scope of the invention, it is intended that all matter contained in the above description shall be interpreted as illustrative and not in a limiting sense.

All references cited in this specification, including patents and patent applications, are hereby incorporated by reference. The discussion of references herein is intended merely to summarize the assertions made by their authors and no admission is made that any reference constitutes prior art. Applicant reserves the right to challenge the accuracy and pertinence of the cited references.

Human Efficacy Data from Using Arachidoninc Acid Analogue

The idea in this invention pertaining to using arachidonic acid analogues has already been tested in humans and proven to work. The 5,6-dehydro arachidonic acid has been synthesized and a topical formulation was developed. The formulation consist of 0.01-1 microgram of 5,6-dehydro arachidonic acid placed in a 1-1000 ratio of DMSO. The formulation was applied under arm pits (topically) to patients suffering from Cystic Fibrosis, psoriasis, ulcerative colitis and Ankylosing Spondylitis. Six patients suffering from Cystic fibrosis were administered this formulation once monthly. All of them showed significant clinical benefit and reduction of symptoms.

Approximately 30 patient with psoriasis applied this formulation to it completely healed their psoriatic lesions on their skin and they remained symptom free for years due to the high affinity of the analogue so the blood cells.

One patients with ulcerative colitis utilized the drug and it 100% reversal of symptomatology and 100% decrease in all ulcerative mediations.

Three patients with Ankylosing Spondylitis have been treated with this drug and it has helped them with the symptoms

What is claimed is:

1. A method of treating a disease mediated by arachidonic acid or an eicosanoid, the method comprising administering to a subject in need thereof an effective amount of an analogue of arachidonic acid or an eicosanoid,
wherein the analogue is selected from the group consisting of an arachidonic acid analogue, an HPETE analogue, an EET analogue, a prostaglandin analogue and an HETE analogue, and
wherein the disease is selected from the group consisting of cancer, ischemic heart disease, psoriasis, cystic fibrosis, Alzheimer's disease, allergy, COPD, rheumatoid arthritis (RA), ulcerative colitis, or Crohn's disease

2. The method of claim 1, the analogue is an arachidonic acid analogue having a triple bond at from one to three of positions selected from the group consisting of C5-C6, C8-C9, C11-C12 and C14-C15.

3. The method of claim 1, wherein the analogue is an HPETE analogue.

4. The method of claim 3, wherein the HPETE analogue is a 15-HPETE analogue having a triple bond at from one to four of positions selected from the group consisting of C5-C6, C8-C9, C11-C12 and C13-C14.

5. The method of claim 3, wherein the HPETE analogue is a 12-HPETE analogue having a triple bond at from one to four of positions selected from the group consisting of C5-C6, C8-C9, C10-C11 and C14-C15.

6. The method of claim 3, wherein the HPETE analogue is a 5-HPETE analogue having a triple bond at from one to four of positions selected from the group consisting of C6-C7, C8-C9, C11-C12 and C14-C15.

7. The method of claim 1, wherein the analogue is an EET analogue.

8. The method of claim 7, wherein the EET analogue is a 5,6-EET analogue having a triple bond at from one to three of positions selected from the group consisting of C8-C9, C11-C12 and C14-C15.

9. The method of claim 7, wherein the EET analogue is an 8,9-EET analogue having a triple bond at from one to three of positions selected from the group consisting of C5-C6, C11-C12 and C14-C15.

10. The method of claim 7, wherein the EET analogue is a 14,15-EET analogue having a triple bond at from one to three of positions selected from the group consisting of C5-C6, C8-C9 and C11-C12.

11. The method of claim 7, wherein the EET analogue is a 11,12-EET analogue having a triple bond at from one to three of positions selected from the group consisting of C5-C6, C8-C9 and C14-C15.

12. The method of claim 1, wherein the analogue is a prostaglandin analogue.

13. The method of claim 12, wherein the prostaglandin analogue is a PGG2 analogue having a triple bond at from one to two positions selected from the group consisting of C5-C6 and C13-C14.

14. The method of claim 12, wherein the prostaglandin analogue is a PGH2 analogue having a triple bond at from one to two positions selected from the group consisting of C5-C6 and C13-C14.

15. The method of claim 1, wherein the analogue is an HETE analogue.

16. The method of claim 15, wherein the HETE analogue is a 5-HETE analogue having a triple bond at from one to four positions selected from the group consisting of C6-C7, C8-C9, C11-C12 and C14-C15.

17. The method of claim 15, wherein the HETE analogue is an 8-HETE analogue having a triple bond at from one to four positions selected from the group consisting of C5-C6, C9-C10, C11-C12 and C14-C15.

18. The method of claim 15, wherein the HETE analogue is a 9-HETE analogue having a triple bond at from one to four positions selected from the group consisting of C5-C6, C7-C8, C11-C12 or C14-C15.

19. The method of claim 15, wherein the HETE analogue is an 11-HETE analogue having a triple bond at from one to four positions selected from the group consisting of C5-C6, C8-C9, C12-C13 and C14-C15.

20. The method of claim 15, wherein the HETE analogue is a 12-HETE analogue having a triple bond at from one to four positions selected from the group consisting of C5-C6, C8-C9, C10-C11 and C14-C15.

21. The method of claim 15, wherein the HETE analogue is a 15-HETE analogue having a triple bond at from one to four positions selected from the group consisting of C5-C6, C8-C9, C11-C12 and C13-C14.

22. The method of claim 15, wherein the HETE analogue is a 19-HETE analogue having a triple bond at from one to four positions selected from the group consisting of C5-C6, C8-C9, C11-C12 and C14-C15.

23. The method of claim 15, wherein the HETE analogue is a 20-HETE analogue having a triple bond at from one to four positions selected from the group consisting of C5-C6, C8-C9, C11-C12 and C14-C15.
